(19) United States
(12) Patent Application Publication
(10) Pub. No.: US 2006/0057203 A1
(43) Pub. Date: Mar. 16, 2006

(54) Modified release formulations of oxcarbazepine and derivatives thereof

(76) Inventors: Marie-Christine Wolf, St. Louis (FR); Oskar Karb, Lorrach (DE); Jean-Daniel Bonny, Fullinsdorf (CH); Stefan Hirsch, Lorrach (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/528,687

(22) PCT Filed: Sep. 19, 2003
(86) PCT No.: PCT/EP03/10475

§ 371 Date: Mar. 21, 2005

(30) Foreign Application Priority Data
Sep. 20, 2002 (GB) 0221956.6

(51) Int. Cl. A61K 9/24

(52) U.S. Cl. 424/472

(57) ABSTRACT

Oral once a day dosage forms comprising oxcarbazepine are disclosed.

### FIELD OF THE INVENTION

The present invention relates to formulations of oxcarbazepine (hereinafter referred to as “the compound of the invention”).

### BACKGROUND OF THE INVENTION

Oxcarbazepine (also referred to herein as OXC) is an anticonvulsant agent. The preparation of oxcarbazepine and its pharmaceutically acceptable salts is described, e.g., in German patent 2,011,087 which is incorporated herein by reference. A commercially advantageous process of manufacturing oxcarbazepine is disclosed in European Patent No. 28 028 wherein oxcarbazepine is obtainable by hydrolysing 5-carbamoyl-10-amino-5H-dibenz[b,f]azepine. For example 5-cyano-5H-dibenz[b,f]azepine is nitrated, the resulting nitro derivative is hydrolysed to the 5-carbamoyl derivative, the nitro moiety is reduced and the reduction product is hydrolysed to oxcarbazepine. This patent is incorporated herein by reference. Oxcarbazepine is indicated for the treatment of partial-onset seizures with or without secondarily generalized tonic-clonic seizures, in both adults and children aged over 6 years, as monotherapy or adjunctive therapy. The compound of the invention has been commercialized for over ten years as an immediate release formulation, e.g. under the registered trademark Trileptal™. Extensive clinical experience has shown that the compound of the invention is a valuable antiepileptic drug for the treatment of adults and children with partial onset seizures both in initial monotherapy, for conversion to monotherapy and as adjunctive therapy.

For monotherapy, oxcarbazepine may be initiated with a dose of 600 mg/day (8-10 mg/kg/day) given in 2 divided doses per day. Therapeutic effects are seen at doses between 600 mg/day and 2400 mg/day. Controlled monotherapy trials in patients not currently being treated with antiepileptic drugs (AEDs) showed 1200 mg/day to be an effective dose; however, a dose of 2400 mg/day has been shown to be effective in more refractory patients converted from other AEDs to oxcarbazepine monotherapy.

For adjunctive therapy, oxcarbazepine may be initiated with a dose of 600 mg/day (8-10 mg/kg/day) given in 2 divided doses per day. Therapeutic responses are seen at doses between 600 mg/day and 2400 mg/day. In children, therapeutic effects were seen at a median maintenance dose of approximately 30 mg/kg/day.

Oxcarbazepine is practically insoluble in water. It is completely (>95%) absorbed and extensively metabolized by reduction to a pharmacologically active metabolite, the monohydroxy derivate of oxcarbazepine (10,11-dihydro-10-hydroxy-5H-dibenz[b,f]azepine-5-carboxamide) referred to herein as MHD. Pharmacokinetic investigations in humans depend on determination of plasma concentrations of MHD on the basis that biotransformation of oxcarbazepine to MHD is almost complete.

The mechanisms by which OXC and MHD exert their anticonvulsant effect are not completely understood but may be partly due to effects on ion flow across neuronal membranes. Specifically, OXC and MHD have been shown to modulate voltage-dependent sodium channels in the brain.

Pharmacokinetics and absorption sites and mechanisms of the compound of the invention are not understood in detail however.

The currently available oral dosage forms, such as film-coated tablets and liquid dosage forms, e.g. 6% oral suspension, of the compound of the invention are suitable for ensuring appropriate concentrations of the compound of the invention in the blood by repeated administration over a prolonged period of treatment.

Despite the merits of the known oral dosage forms, there remains a need to improve upon existing oral dosage forms of the compound of the invention. One of the problems that may occur is the fluctuation of blood levels of the compound of the invention on repeated administration which may be associated with side effects.

After exhaustive testing surprisingly we have now invented oral dosage forms of the compound of the invention with advantageous properties, e.g. producing constant plasma levels of MHD over 24 hours.

### SUMMARY OF THE INVENTION

The invention provides in one aspect an oral dosage form comprising oxcarbazepine adapted to be administered once a day (hereinafter “oral dosage form of the invention”).

Oral dosage forms according to the invention may represent a considerable advantage over currently marketed oral dosage forms in that they are more convenient and/or safer for patients to use and increase the patient's compliance to therapy. The patient has to take the oral dosage form of the invention only once a day.

In another aspect the present invention provides an oral dosage form comprising oxcarbazepine which, when administered once a day, is released to produce constant MHD plasma levels over 24 hours.

### DESCRIPTION OF THE INVENTION

The following embodiments have particularly interesting properties.

In one embodiment of the invention, the oral dosage form of the invention consists of a tablet core and a coating wherein the core comprises oxcarbazepine, optionally, a filler, and at least one further excipient selected from cellulose ethers, carboxyvinyl polymer of acrylic acid cross linked with alkyl ethers of sucrose or pentaerythritol and polymethacrylates.

In one embodiment (hereinafter variant 1) the dosage form has 80% or greater release of the oxcarbazepine dose within 1 hour, indicated in standard in vitro dissolution tests, e.g. effected by use of the apparatus 2 (Rotary Paddle) of the USP at 37 degrees Celsius in water at a stirring rate of 60 rpm using sodium dodecyl sulphate as a solubilizing agent at a concentration of 1% for 600 mg dosage form and based on a mean of 6 or more e.g. 10. dosage forms hereinafter referred to as “in vitro oxcarbazepine test dissolution conditions of the invention”).

Examples of minimum releases rates are:
a) 68% or
b) 80%
within 0.5 hours (after the start of the experiment).

Examples of maximum release rates are:
a) 84% or
b) 91%
within 1 hour (after the start of the experiment)

Examples of typical release rates are

0.5 hours from 70 to 85% or

1 hour from 80 to 95%

In another embodiment (hereinafter variant 2) the dosage form has 40% or more release at 4 hours or 80% or less release of the dose at 4 hours, as indicated in standard in vitro dissolution tests, e.g. in vitro oxcarbazepine test dissolution conditions of the invention. Preferably the release is not more than 80% after 8 hours

Examples of minimum releases rates are:
a) 30 or
b) 37%
within 4 hours (after the start of the experiment).

Examples of maximum release rates are:
a) 75 or
b) 80%

within 8 hours (after the start of the experiment).

Examples of typical release rates are

a) 12 hours from 75 to 90%

b) 8 hours from 60 to 70%

In another embodiment (hereinafter variant 3) the dosage form releases oxcarbazepine at a constant release rate for 4 hours or more, as indicated in standard in vitro dissolution tests, e.g. in vitro oxcarbazepine test dissolution conditions of the invention, e.g. from an oral osmotic system.

Preferably the release is about 80% after 8 hours.

Such a constant release may be typically that associated with an oral osmotic system, e.g. varying by less than 30% per hour over e.g. 4 hours.

Examples of typical release rates are:
a) 12 hours 80 to 90%
b) 8 hours 75 to 90%

The absorption profile of the compound of the invention namely. OXC and. MHD may be quantified by making Area Under the Curve (AUC) measurements on single doses or at steady state.

Constant plasma levels of MHD indicate that the plasma levels of MHD show low fluctuation indices. Minimum concentrations (Cmin) and maximum plasma concentrations (Cmax) of MHD may be kept in a small range. To measure the fluctuation between Cmin and Cmax the MHD levels are measured at the steady state and the fluctuation index is calculated: (Cmax−Cmin)/Cav wherein Cmax is the maximum concentration, Cmin is the minimum concentration, Cavs is the average concentration observed in a certain time interval e.g. 24 hours at steady state.

Typically the fluctuation index for a 600 mg dose of immediate release oxcarbazepine given once a day is about 40%. Thus oral dosage forms of the invention may e.g. have from about 20% to about 60%, preferably 30% to 50% as the fluctuation index value for a 600 mg dose of immediate release oral dosage form and such value being “low fluctuation index”.

Low fluctuation of Cmin and Cmax may avoid peak values of MHD plasma levels, which can be toxic for the patient. Lower fluctuation may provide better tolerability and safety for the patient treated with oxcarbazepine.

We have now found that the compound of the invention administered twice a day resulted in an increase of MHD plasma level in the first three hours and then in a decrease till the oxcarbazepine had to be re-administered after 12 hours. Due to this fluctuation if plasma levels decreased until the therapeutically ineffective levels were reached the anticonvulsive action of the compound of the invention may not be maintained and seizures occurred as severe side effects. This fluctuation may be reduced by the oral dosage forms of the invention by achieving constant plasma levels of MHD over 24 hours when the drug is administered once a day.

Conventional rapid release forms of the compound of the invention may not be suitable to achieve constant plasma levels of MHD over 24 hours when administered once a day. The inventors have now found oral dosage forms that provide a sustained release. These sustained release forms may be capable of providing constant plasma levels over 24 hours when administered once a day and which are therapeutically effective.

Galenical Principles

A wide range of sustained release galenical principles may be used to achieve once-a-day administration. Sustained release systems include any drug delivery system that achieves delayed or slow release of drug over an extended period of time. Preferred sustained release systems are as follows:
I) Erodible matrix systems
II) Coated systems.
III) Osmotic systems

In one aspect of the invention the sustained release system may be in the form of a matrix. Excipients may be formed in a matrix which modifies the release of the compound of the invention dispersed within said matrix.

In another embodiment the compound of the invention is coated with a polymer which modifies the release of the compound of the invention and is referred to herein as a coated system. The compound of the invention may be mixed with varying amounts of excipients before coating.

In a further embodiment the compound of the invention is formulated as an oral osmotic dosage form, an osmotic system, designed in use to imbibe water and release the compound of the invention at a constant rate into aqueous fluids.

If desired immediate release systems of the compound of the invention may be mixed with sustained release systems. For example disintegrating and erodable tablets may be formulated to reduce food effects. Quick/slow tablets may be formulated to obtain constant plasma levels without loss in AUC. Combinations of immediate release systems and release systems may provide constant plasma levels of MHD.

If desired for modifying the release of the compound of the invention, the compound may be mixed with a surfactant preferably with an HLB value of 10 or more and especially an ionic surfactant e.g. sodium dodecyl sulfate (SDS) or another surfactant as mentioned hereinafter.

Coating materials which modify the release of the compound may be used, especially methacrylates e.g. trimethyl ammonium methacrylate and cellulose ethers e.g. ethylcellulose.

Osmotic systems may comprise a core comprising the compound of the invention an excipients, a semi permeable wall around said core permeable to water and gastrointestinal fluid and a hole through said semi permeable wall connecting said core with external environment.

Immediate release systems, e.g. comprising the compound of the invention, preferably in finely ground form, having a median particle size of approximately from 2 to 12 μm and further excipients, may be combined with sustained release formulations.

A combination may be a double-layer tablet comprising an immediate release system and a matrix system wherein the compound of the invention e.g. oxcarbazepine in admixture with a surfactant e.g. sodium dodecyl sulfate (SDS) also known as sulfuric acid monododecyl ester sodium salt.

A double-layer tablet may comprise two doses of the compound of the invention e.g. oxcarbazepine, one part being adapted to provide a sustained release dose and another part adapted to provide an immediate release dose. By immediate release is meant release of at least 90% of the dose within 0.5 hours and 100% of the dose within 1.5 hours under in vitro oxcarbazepine test dissolution conditions of the invention. By sustained release is meant release of at least 60% preferably at least 75% and not more than 85% of the dose within 0.5 hours, at least 80% and not more than 95% of the dose within 1 hour, at least 95% of the dose within 3 hours under in vitro oxcarbazepine test dissolution conditions of the invention.

A double layer tablet may comprise a tablet having separate layers with different with different release profiles. One inner layer may comprise a formulation of the compound of the invention comprising a core adapted to provide a sustained release dose of the compound of the invention and the outer layer may be adapted to provide immediate release of the compound of the invention.

A further embodiment of the invention comprises a combination of an immediate release system and a coated system wherein the compound of the invention is coated by methacrylate.

Conveniently solid dosage form of the invention may be produced by compressing the compound of the invention with e.g. conventional tabletting excipients to form a tablet core using conventional tabletting processes and subsequently coating the core. The tablet cores can be produced using conventional granulation methods, for example wet or dry granulation, with optional comminution of the granules and with subsequent compression and coating. Granulation methods are described, for example, in R. Voigt, Lehrbuch der Pharmazeutischen Technologie, Verlag Chemie, 6^{th} edition, pages 156-169. Granules may be produced in a manner known per se, for example using wet granulation methods known for the production of “built-up” granules or “broken-down” granules. Methods for the formation of built-up granules may comprise, for example simultaneously spraying the granulation mass with granulation solution and drying, for example in a drum granulator, in pan granulators, on disc granulators, in a fluidized bed, by spray-drying or spray-solidifying, or operate discontinuously, for example in a fluidized bed, in a batch mixer or in a spray-drying drum.

Preferred are methods for the production of broken-down granules, which may be carried out in batches. The granulation mass may first form a wet aggregate with the granulation solution, which aggregate is then comminuted or formed into granules of the desired particle size and the granules then being dried. Suitable equipment for the granulation step are planetary mixers, low and high shear mixers, wet granulation equipment including extruders and spheronisers.

The granulation mass may comprise the comminuted, preferably ground compound of the invention and excipients.

Depending on the method used, the granulation mass may be in the form of a premix or e.g. may be obtained by mixing the compound of the invention with one or more excipients. The wet granules are preferably dried, for example in the described manner by tray drying or in a fluidised bed.

The compound of the invention displays a tendency towards discolouration upon storage and coatings, e.g. a single or a double film coating may be beneficial in masking any discolouration. Accordingly the invention provides in another of its aspects, a solid oral dosage form which is stable to discolouration. Preferably oral dosage forms that are stable to discolouration remain so stable for at least 3 years storage at a temperature of 25° C. and 60% r.h.

It may be beneficial in masking discolouration, to use colouring agents, e.g pigments in oral dosage forms of the invention. In the case of a tablet, colouring agents may be mixed with the compound of the invention and tabletting excipients in the core or they may alternatively be placed solely in a coating composition, or both in the core and in the coating composition.

In a preferred embodiment according to the invention theoral dosage form may be in the form of a film-coated tablet. Coveniently the film is soluble in stomach juices and may be ca 20 mg per 600mg compound of the invention dosage form.

Oral dosage forms according to the invention may contain, in addition to the compound of the invention, conventional excipients depending on the exact nature of the formulation. Suitable categories of excipients include fillers, lubricants, film coating agents, binders, glidants, solubilizers, surface-active substances and disintegrants.

Excipients disclosed in the literature, as for instance in Fiedler's “Lexikon der Hilfstoffe”, 4th Edition, ECV Aulendorf and “Handbook of Pharmaceutical Excipients”, Wade and Weller, Third Edition (2000), the contents of which are incorporated herein by reference, may be used in the pharmaceutical compositions according to the invention. Conveniently the excipients comprise less than 40% of the weight of the dosage form.

We have found that certain excipients exhibit especially interesting properties in oral dosage forms of the invention e.g.

a) cellulose ethers, such as

i) Hydroxypropyl methylcellulose, e.g.
Preferred are Methocel K 100 and cellulose HPM 100T of viscosities of 80000 to 120000 mPa s, e.g. in a weight ratio of from about 1:3 to about 1:8.
Methocel HG which has a 2 percent aqueous viscosity of approximately 4000 mPa s, a methoxyl content of 26 to 30%, and a hydroxypropyl content of 7 to 12%.
CR grade Methocel E-4M, which has a 2 percent aqueous viscosity of approximately 4,000 mPa s, a number average molecular weight of approximately 90,000, a methoxyl content of 28.0 to 30.0%, and a hydroxypropoxyl content of 7.0 to 12.0% or equivalent, e.g. 10- 20% by tablet.
Methocel E-50 Premium, which has a 2 percent aqueous viscosity of approximately 50 mPa s, a number average molecular weight of approximately 20,000, a methoxyl content of 28.0 to 30.0%, and a hydroxypropoxyl content of 7.0 to 12.0%or equivalent (e.g. 10-20% by weight per tablet).
A preferred weight ratio of total hydroxypropylmethyl cellulose to the compound of the invention is from about 1: 10 to about 1:20.
Hydroxypropyl methyl cellulose (HPMC) polymers may be used as matrix components modifying the release of the drug, either alone or in combination with other materials. Oral doasage forms of the invention containing HPMC polymers may prolong drug release by forming a gelatinous matrix upon exposure to the aqueous medium of the stomach which prevents or delays ingress of the aqueous medium of the stomach into the dosage form and thereby preventing its rapid disintegration. The gel matrix may be formed as a result of hydration of the HPMC polymer. Insignificant instability problems during storage of the oral dosage form of the present invention comprising oxcarbazepine, excipients in combination with HPMC may occur.
A preferred excipient to use as a matrix component is a cellulose ether product such as: methylcellulose and hypromellose. Such hypromellose products may be made wherein propylene oxide is used in addition to methyl chloride to obtain hydroxypropyl substitution on the anhydroglucose units in cellulose. This substituent group, —OCH2CH(OH)—CH3, contains a secondary hydroxyl on the number two carbon and may also be considered to form a propylene glycol ether of cellulose. These products possess varying ratios of hydroxypropyl and methyl substitution, a factor which influences organic solubility and the thermal gelation temperature of aqueous solutions.
Viscosities are preferably from 1000 to 10000 cps.
Such products include Methocel products available from Dow Chemical company USA, An alternative is an ethyl cellulose such as Aquacoat available as a 30 wt. % ethylcellulose dispersion from FMC.
Preferably the weight ratio of cellulose ether product to compound of the invention e.g. the compound of the invention is from about 1:1 to about 1:20.
Hydroxypropylmethyl cellulose (as mentioned above) is a preferred excipient, for example the quality of Cellulose HPM 603 which has a viscosity of about 3 mPa s, available e.g. as Pharmacoat® 603 (Fiedler, loc.cit., p. 1172). It may act as a binder. Cellulose derivatives such as hydroxypropylmethylcellulose, preferably have a molecular weight of from 10 000 to 1 500 000 Daltons.

ii) Ethylcellulose, e.g. Ethocel Premium 7 cps, which has a 2 percent aqueous viscosity of approximately 7 cps and an ethoxyl content of 44.0 to 51.0% or equivalent e.g. 7-10%. Hence, the present invention provides an oral dosage form of the invention wherein a cellulose ether is employed which is ethyl cellulose, preferably having a weight ratio of total ethyl cellulose to oxcarbazepine from about 1:10 to about 1:20.

iii) Hydroxypropylcellulose, e.g. Klucel LF, which has a 5% viscosity of approximately 100 cps, and a hydroxypropoxyl content of approximately 54 to 77% or equivalent (e.g. 0.5-5% by weight per tablet) or hydroxyethyl cellulose (HEC).

Hydroxypropyl cellulose may be e.g. hydroxypropyl cellulose having a hydroxypropyl content of 5 to 16% by weight and a molecular weight of from 80,000 to 1,150,000, more particularly 140,000 to 850,000

b) Carbomer; carboxyvinyl polymer of acrylic acid cross linked with alkyl ethers of sucrose or pentaerythritol), e.g. Carbopol 934P, which has a 0.5% aqueous viscosity of approximately 37,000 mPas. (e.g. 0.01-1% by weight per tablet).

c) Polysorbate 80, e.g. Tween 80, which is a sorbitan, mono-9-octadecanoate, poly(oxy-1,2-ethanediyl) derivative. (e.g. 1-5% by weight per tablet).

d) Polymethacrylates, such as trimethylammonium methacrylate, in particular the pharmaceutical polymethacrylates marketed under the brand Eudragit™.

Examples of other binders include
starches, e.g., potato starch, wheat starch, corn starch; e.g. having a molecular weight of from 30 000 to 120 000,
polyvinyl pyrrolidone, e.g., Povidone, especially having a mean molecular weight of approximately 1000 and a degree of polymerisation of approximately from 500 to 2500.
and polymethylacrylates, having a molecular weight of 2 100 000 Daltons, for example copolymers of acrylic or
methacrylic acid esters, known as Eudragit RL 30D (Handbook of Pharmaceutical Excipients loc.cit., p. 402).

Microcrystalline cellulose is preferably present. It may be used as a filler. Examples include the Avicel® type (FMC Corp.), for example of the types AVICEL PH101, 102, 105, RC581 or RC 591 (Fiedler loc.cit., p. 216), Emcocel® type (Mendell Corp.) Elcema® type (Degussa), Filtrak® type, Heweten® type or Pharmacel®.

Hence, the present invention provides an oral dosage form of the invention comprising as a filler microcrystalline cellulose.

Another preferred filler is for example a pulverulent filler especially optionally having flow-conditioning properties, including carbohydrates, such as sugars, sugar alcohols, starches or starch derivatives, for example lactose, dextrose, saccharose, glucose, sorbitol, mannitol, xylitol, potato starch, maize starch, rice starch, wheat starch or amylopectin, tricalcium phosphate or calcium hydrogen phosphate.

Preferably the filler is present in a weight ratio of microcrystalline cellulose or a filler to the compound of the invention e.g. oxacarbazepine from about 1:10 to about 1:30. Polyvinyl-polypyrilidone is preferably present. Conveniently it functions as a disintegrant. A preferred example is a crosslinked polyvinylpyrrolidone, e.g. crospovidones, e.g. Polyplasdone® XL (Fiedler loc.cit., p. 1245) and Kollidon® CL disintegrant. Examples of other disintegrants include: (i) natural starches, such as maize starch, potato starch, and the like, directly compressible starches, e.g. Sta-rx@ 1500, modified starches, e.g. carboxymethyl starches and sodium starch glycolate, available as Primojel®, Explotab®, Explosol®, and starch derivatives such as amylose; (ii); crosslinked sodium carboxymethylcellulose, available as e.g. Ac-di-sol®, Primellose®, Pharmacel® XL, Explocel®, and Nymcel® ZSX; (iii) alginic acid and sodium alginate; (iv) methacrylic acid-divinylbenzene copolymer salts, e.g. Amberlite® IRP-88, and vi).magnesium aluminium silicate, bentonite, alginic acid and alginates.

Colloidal silicas e.g. Aerosil 200 (Fiedler, loc.cit., p117) may be preferably present. These may act as a glidant. Examples of other glidants include: silica, magnesium trisilicate, powdered cellulose, starch, talc and tribasic calcium phosphate.

Magnesium stearate is a preferred excipient. It may function as a lubricant. Examples of other lubricants include: calcium stearate, zinc stearate, talc, polyethylene glycol, stearic acid, sodium benzoate, sodium dodecyl sulfate, also know as sulphuric acid monododecyl ester sodium salt available as Duponol C (Fiedler loc.cit., p. 517), mineral oil, and polyoxyethylene monostearate. A combination of lubricants may also be used.

An alkyl sulfate is preferably present. It may function as a surfactant. Preferred examples are sodium dodecyl sulfate (n-tetradecyl sulfate, n-hexadecyl sulfate or n-octadecyl sulfate for example sodium, potassium or magnesium n-dodecyl sulfate. Sodium lauryl sulphate (SDS) for example is available as Duponol C (Fiedler loc.cit., p. 517).

Other surfactants of the anionic type include an alkyl ether sulfate type, for example sodium, potassium or magnesium n-dodecyloxyethyl sulfate, n-tetradecyloxyethyl sulfate, n-hexadecyloxyethyl sulfate or n-octadecyloxyethyl sulfate, or of the alkanesulfonate type, for example sodium, potassium or magnesium n-dodecanesulfonate, n-tetradecanesulfonate, n-hexadecanesulfonate or n-octadecanesulfonate.

If desired non-ionic surfactants may be used of the fatty acid polyhydroxy alcohol ester type, such as sorbitan monolaurate, monooleate, monostearate or monopalmitate, sorbitan tristearate or trioleate, polyoxyethylene adducts of fatty acid polyhydroxy alcohol esters, such as polyoxyethylene sorbitan monolaurate, monooleate, monostearate, monopalmitate, tristearate or trioleate, polyethylene glycol fatty acid esters, such as polyoxyethyl stearate, polyethylene glycol 400 stearate, polyethylene glycol 2000 stearate, especially ethylene oxide/propylene oxide block polymers of the Pluronics® (BWC) or Synperonic® (ICI) type. Examples of other surfactants include: phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2).

“Polyoxyethylated” means that the substances in question contain polyoxyethylene chains, the degree of polymerization of which generally lies between 2 and 40 and in particular between 10 and 20.

Preferably the weight ratio of surfactant to the compound of the invention is from about 1:50 to about 1:500.

A granulate of the compound of the invention may be coated. Preferred coating materials are methacrylates such as Eudragit RTM, RS and RL or ethyl cellulose such as Aqucoat. Preferably the weight ratio is from about 1:10 to 1:20.

Suitable coating materials include those materials conventionally used in coating tablets, granules and the like. In one group of embodiments the coating is water soluble. In another group of embodiments the coating is gastric juice resistant but soluble in intestinal juices. Coating materials may be used in admixture with other excipients, conventional in coating formulations, for example silicon dioxide, for example synthetic amorphous silicic acid of the Syloid® type (Grace), for example SYLOID 244 FP, for example sorbates or plasticisers e.g. triethyl citrate, e.g. Citroflex® (Pfizer), triacetin, various phthalates, e.g. diethyl or dibutyl phthalate, mixed mono- or di-glycerides of the Myvacet® type (Eastman), e.g. MYVACET 9-40, the polyethylene glycols, for example having a molecular weight of approximately from 6000 to 8000, and also ethylene oxide/propylene oxide block copolymers of the Pluronic® (BASF) or Synperonic (ICI) type, pulverulent mould release agents, for example magnesium trisilicate, starch or synthetic amorphous silicic acid.

In particular, coating materials suitable for use in the practice of the invention include but are not limited to polymer coating materials, such as cellulose acetate phthalate, cellulose acetate trimaletate, hydroxy propyl methylcellulose phthalate, polyvinyl acetate phthalate, ammonio methacrylate copolymers, poly acrylic acid and poly acrylate and methacrylate copolymers, polyvinyl acetaldiethylamino acetate, hydroxypropyl methylcellulose acetate succinate, shellac; hydrogels and gel-forming materials, such as carboxyvinyl polymers, sodium alginate, sodium carmellose, calcium carmellose, sodium carboxymethyl starch, poly vinyl alcohol, hydroxyethyl cellulose, methyl cellulose, gelatin, starch, and cellulose based cross-linked polymers in which the degree of crosslinking is low so as to facilitate adsorption of water and expansion of the polymer matrix, hydoxypropyl cellulose, hydroxypropyl methylcellulose, polyvinylpyrrolidone, crosslinked starch, microcrystalline cellulose, chitin,, pullulan, collagen, casein, agar,, sodium carboxymethyl cellulose, polyvinylpyrrolidone (m. wt. .about.10k-360k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin, polysaccharides such as, acacia, karaya, tragacanth, algins and guar, polyacrylamides, diesters of polyglucan, crosslinked polyvinyl alcohol and poly N-vinyl-2-pyrrolidone, sodium starch glucolate (e.g. Explotab®; Edward Mandell C. Ltd.); hydrophilic polymers such as polysaccharides,, sodium or calcium carboxymethyl cellulose, hydroxypropyl cellulose, hydroxyethyl cellulose, nitro cellulose, carboxymethyl cellulose, cellulose ethers, polyethylene oxides (e.g. Polyoxe®, Union Carbide), methyl ethyl cellulose, ethylhydroxy ethylcellulose, cellulose acetate, cellulose butyrate, cellulose propionate, gelatin, collagen, starch, maltodextin, pullulan, polyvinyl pyrrolidone, polyvinyl alcohol, polyvinyl acetate, glycerol fatty acid esters, sorbitan esters, natural gums, lecithins, pectin, alginates, ammonia alginate, sodium, calcium, potassium alginates, propylene glycol alginate, and gums such as arabic, locust bean, carrageens, guar, xanthan, scleroglucan and mixtures and blends thereof. As will be appreciated by the person skilled in the art, excipients such as plasticisers, lubricants, and the like may be added to the coating. Suitable plasticisers include for example acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, dilsononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate.

The compound of the invention and methods for its preparation are well known in the art. Its manufacture and therapeutic use are described in German Auslegeschrift 2 011087 which is incorporated herein by reference. A commercially advantageous process of manufacturing the compound of the invention is disclosed in European Patent No. 28 028 wherein the compound of the invention is obtainable by hydrolysing 5-carbamoyl-10-amino-5H-dibenz[b,f]azepine. For example 5-cyano-5H-dibenz[b,f]azepine is nitrated, the resulting nitro derivative is hydrolysed to the 5-carbamoyl derivative, the nitro moiety is reduced and the reduction product is hydrolysed to oxcarbazepine. This patent is incorporated herein by reference. Conveniently the manufacturing process disclosed in European Patent Application No. 1915203.2 wherein the compound of the invention is obtainable by hydrolysing 5-carbamoyl-10-(C₁₋₄ e.g C₁) alkoxy-5H-dibenz[b,f]azepine. For example 10-alkoxy-5H-dibenz[b,f] azepine (obtained through a cyclisation step) in is carbamoylated and the alkoxy moiety is hydrolised to oxcarbezepine.

It will be appreciated that the present invention encompasses solid oral dosage forms of the invention with any of the above excipients alone or in connection.

The invention provides in further aspect a compressed tablet of an ovaloid shape. The tablet may be small in dimension e.g. 10 to 20 mm in length, preferably 15 to 19 mm; 5 to 10 mm in width, preferably 6 to 8 mm.

Preferably the compound of the invention e.g. oxcarbazepine is in finely ground form, especially in mono-modal form, having a median particle size of approximately from 2 to 12 μm, preferably 4 to 12 μm, more preferably 4 to 10 μm with a maximum residue on a 40 μm sieve of up to 5%, e.g. 2%.

Unlesss otherwise indicated, all percentages are weight by weight.

Clinical bioavailabilty trials may be effected in conventional manner. For example they may be effected over 7 or more days using a 600 mg dose of the compound of the invention. Conveneiently at least 6, e.g 10, subjects are used.

During a first 7 day period subjects will be given one of the oral dosage forms of the invention under fasted conditions and during the second period subjects will be given the same treatment under fed conditions. Subjects will fast overnight for a minimum of 10 hours on the evening prior to the first dosing of the compound of the invention (period 1).

Following dosing (e.g 600 mg of the compound of the invention) at e.g breakfast time, pharmacokinetic blood samples may be drawn and used for the oxcarbazepine and MHD assays at adequate time intervals (e.g 0.5, 1, 2, 3, 4, 6, 8, 10, 12, 14, 16, 18, 20, 22, 24, 32 and 48 hours after administration).

Such trials are referred to hereinafter as method A (single dose) or method B (steady state) respectfully.

We have found that the ratio of oxcarbazepine to MHD plasma-concentration is of about 90:10.

In a single dose study (one dose of 600 mg oxcarbazepine at fasted conditions and MHD levels quantified) the AUC (0 to 48 hours) is 700 to 800 h*micromol/L preferably about 750 h*micromol/L, Cmax 10 to 30 micromol/L preferably from 20 to 25 micromol/L (Table A) Conveniently the AUC in the fasted state is more than 500 h*micromol/L.

Preferably the ratio Cmax/C24 h for MHD levels (maximum concentration of MHD levels versus concentration of MHD levels after 24 hours) is less than 2.3, e.g from 1 to 1.5 (Table B).

The exact dose of and the particular oral dosage forms of the invention to be administered depend on a number of factors, e.g. the condition to be treated, the desired duration of treatment and the rate of release of the compound of the invention.

Preferred regimens according to the invention include for monotherapy, e.g. 600 mg or e.g. 1200 mg once a day. Doses from 600 to 2400 mg/day of the compound of the invention are well tolerated.

The oral dosage forms of the invention are useful for their anticonvulsive action and are useful as monotherapy or as adjunctive therapy in the control, prevention or treatment of seizure, e.g. resulting from the onset of epilepsy, status epilepticus, cerebrovascular disorders, head injury and alcohol withdrawal. The oral dosage forms of the invention show excellent clinical efficacy and tolerability as indicated in standard animal and clinical trials. Such clinical trials may be effected in conventional manner e.g in a single blind or double blind, randomized, crossover manner in adults, children, and the elderly.

Clinical efficacy may be observed by a decrease in the mean frequency of tonic seizures and tonic-clonic seizures compared with the same dose of the compound of the invention administered twice a day e.g as Trileptal, especially during the initial dosing period.

Side effects may be recorded in conventional manner using score-cards or by laboratory monitoring.

The compositions of the invention show e.g

a) less or frequent and other severe drug interactions such as hyponeutrapenia/hyponatraemia e.g in patients having renal disease, taking medication which may lower serum sodium levels (such as diuretics, oral contraceptives or nonsteroidal anti-inflammatory drugs);

b) decreased cutaneous hypersensitivity reactions e.g drug rash rate in sensitive subjects

c) less central nervous system side-effects (eg, dizziness, headache, diplopia, sedation, somnolence, headache, and ataxia) and

d) less gastrointestinal system side-effects (eg, nausea and vomiting)

e) less menstrual cycle influences on seizure activity (catamenial epilepsy), and

f) fewer pharmacokinetic changes during pregnancy,

Clinical trials may suggest a faster introduction (e.g on switching from other anti-epileptic medication) is indicated than with immediate release of the compound of the invention e.g. 150 mg day one, then 300 mg daily, increased by 300 mg weekly both for monotherapy and adjuvant therapy.

A typical trial may be effected over one or two months and the composition of the invention may be administered once-a-day e.g at breakfast or evening time.

A double-blind, placebo-controlled, randomized, 28-week trial may assess the efficacy and tolerability of the compound of the invention as an oral dosage form of the invention at doses of 600, 1200, and 2400 mg as adjunctive therapy in patients with uncontrolled partial seizures.

Co-medication may be administered to women as 50 micro g estradiol or an equivalent dose of ethinyloestradiol or levonorgestrel in a combined oral contraceptive pill including progestogens. There may be a lower incidence of breakthrough bleeding and contraceptive failure

A trial in children ( aged e.g from 2 to 12 years) may assess the efficacy and toxicity of of the compound of the invention (median dose, 30 mg/kg/d) as adjunctive therapy for partial seizures.

Preferably a 600 mg oxcarbazepine dose is used.

Whereas oral dosage forms according the invention may be in the form of solid oral dosage forms, e.g. capsules, powders, or suspensions, it is preferred if oral dosage forms are in the form of tablets.

The oral dosage form according the invention may also be used for the treatment of other disorders, e.g. those mentioned herein.

In a further aspect the invention provides
a) Use of oxcarbazepine for the manufacture of an oral dosage form medicament to be administered to a patient once a day wherein oxcarbazepine is released to produce a constant profile over 24 hours, in particular for the treatment of epilepsy.
b). A method of orally administering of the compound of the invention e.g for the treatment of epilepsy, said method comprising orally administering to a patient in need of oxcarbazepine therapy once-a-day an oral dosage form of the invention.
c). A method of reducing the variability of bloavailability levels of cyclosporin A for patients during oxcarbazepine therapy, said method comprising orally administering to a patient in need of oxcarbazepine therapy an oral dosage form of the invention.

Following is a description by way of example only of compositions and processes of the invention. In all Examples the compound of the invention is in extra fine mono modal form as defined above.

### EXAMPLE A

### Variant 1—Faster Matrix System

| Formulation | (mg) |
|---|---|
| Tablet Core: | |
| Compound of the invention | 600.0 |
| Avicel PH 102 | 131.2 |
| Cellulose HPM 603 | 16.8 |
| Methocel 60 HG 4000 CP | 45.0 |
| Polyvinyl-polypyrrolidone XL | 30.0 |
| Aerosil 200 | 3.0 |
| Magnesium stearate | 8.0 |
| Weight of the core | 834.0 |
| Coating: | |
| Iron Oxide Yellow | 0.84 |
| TiO2 | 1.25 |
| PEG4000 | 1.67 |
| Cellulose HPM 603 | 16.70 |
| Talc | 2.93 |
| Tablet weight | 857.385 |

a) A pre-mix was prepared containing the compound of the invention, Avicel PH 102 and Cellulose HPM 603.
b) The pre-mix was granulated using a high-shear mixer (e.g. Aeromatic GP65) by wet granulation.
c) The resulting granulation was screened using e.g. a Quadracomill and
d) dried using a fluid bed dryer (e.g. Aeromatic MP3/4).
e) Polyvinyl Polypyrrolidone XL, the rest of Avicel PH102, Methocel 60HG 4000 CP and Aerosil 200 were screened with the dried granulation using a mill (e.g Frewitt) equipped with 1 mm mesh and
f) mixed using a bin blender (e.g. Turbula).
g) Magnesium stearate was screened through a hand screen (0.8 mm mesh) and added.
h) The final blend was mixed using a bin blender (e.g. Turbula).

The final blend was compressed using e.g. a Killian LX18 tabletting press. The tablets were then filmed using e.g. a pan coater Glatt GC750.

Examples of typical release rates are

1 hour from 90 to 95% e.g 95%

under in vitro oxcarbazepine test dissolution conditions of the invention

### EXAMPLE B

### Variant 1—Distegrating Tablet with Faster Encapsulated Granulate System

| Formulation | (mg) |
|---|---|
| Tablet Core: | |
| Compound of the invention | 600.0 |
| Aquacoat ECD30 | 90.0 |
| Avicel PH 102 | 150.0 |
| Croscarmellose sodium (Na- | 75.0 |
| CMC XL) | |
| Aerosil 200 | 2.8 |
| Magnesium stearate | 4.5 |
| Weight of the core | 922.3 |
| Coating: | |
| Iron Oxide Yellow | 0.86 |
| TiO2 | 1.30 |
| PEG4000 | 1.73 |
| Cellulose HPM 603 | 17.25 |
| Talc | 3.02 |
| Tablet weight | 946.46 |

a) The compound of the invention was granulated with a 30% dispersion of Aquacoat ECD30 using a high shear mixer (e.g. Aeromatic GP65).
b) The wet granulate was screened using a mill (e.g. Quadrocomill),
c) dried using a fluid bed dryer (e.g. Aeronmatic MP3/4) and
d) screened using e.g. a Frewitt.

The granulation, screening and drying steps were repeated twice in order to obtain dry substance coated by Aquacoat ECD 30.
e) Avicel PH 102, croscarmellose sodium and Aerosil 200 are screened with the dried granulation using a mill (e.g Frewitt) equipped with 1 mm mesh and
f) mixed using a bin blender (e.g. Turbula).
g) Magnesium stearate was screened through a hand screen (0.8 mm mesh) and added.
h) The final blend was mixed using a bin blender (e.g. Turbula).

The final blend was compressed using e.g. a Killian LX18 tabletting press. The tablets were then filmed using a pan coater e.g. Glatt GC750.

Examples of typical release rates are

2 hours from 92 to 96%, e.g 94%

under in vitro oxcarbazepine test dissolution conditions of the invention

### EXAMPLE C

### Variant 1—Disintegrating Tablet with Encapsulated Granulate System

| Formulation | (mg) |
|---|---|
| Tablet Core: | |
| Compound of the invention | 600.0 |
| Trimethyl ammonium | 90.0 |
| methacrylate, Eudragit RL30D | |
| Avicel PH 102 | 150.0 |
| Croscarmellose sodium (Na- | 75.0 |
| CMC XL) | |
| Aerosil 200 | 2.8 |
| Magnesium stearate | 4.5 |
| Weight of the core | 922.3 |
| Coating: | |
| Iron Oxide Yellow | 0.86 |
| TiO2 | 1.30 |
| PEG4000 | 1.73 |
| Cellulose HPM 603 | 17.25 |
| Talc | 3.02 |
| Tablet weight | 946.46 |

a) The compound of the invention was granulated with a 30% dispersion of Eudragit RL30D using a high shear mixer (e.g. Aeromatic GP65).
b) The wet granulate was screened using e.g. a Quadrocomill,
c) dried using a fluid bed dryer (e.g. Aeromatic MP3/4) and
d) screened using e.g. a Frewitt.

The granulation, screening, drying steps were repeated twice in order to obtain obtain dry substance coated by Eudragit RL30D.
e) Avicel PH 102, croscarmellose sodium and Aerosil 200 were screened with the dried granulation using a mill (e.g Frewitt) equipped with 1 mm mesh and mixed using a bin blender (e.g. Turbula).
f) Magnesium stearate, 4.5 mg, was screened through a hand screen (0.8 mm mesh) and added.
g) The final blend was mixed using a bin blender (e.g. Turbula).

The final blend was compressed using e.g. a Killian LX18 tabletting press. The tablets were filmed using a pan coater e.g. Glatt GC750.

Examples of typical release rates are

2 hours from 91 to 98%, e.g 95%

under vitro oxcarbazepine test dissolution conditions of the invention

### EXAMPLE D

### Variant 2—300 mg Matrix (MR) Layer with 300 mg Immediate Release(IR) Layer

| | (mg) | (mg) |
|---|---|---|
| Formulation | MR layer | IR-layer |
| Tablet Core: | | |
| Compound of the invention | 300.0 | 300.0 |
| Duponol C | 3.0 | |
| Avicel PH 102 | 62.6 | 65.6 |
| Cellulose HPM 603 | 8.4 | 8.4 |
| Cellulose HPM 100T | 36.9 | |
| Aerosil 200 | 1.5 | 1.6 |
| Magnesium stearate | 4.0 | 4.4 |
| Crosspovidone | | 20.0 |
| Weight of the core | 416.4 | 400.0 |
| Coating: | | |
| Iron Oxide Yellow | | 0.53 |
| TiO2 | | 0.79 |
| PEG4000 | | 1.05 |
| Cellulose HPM 603 | | 10.50 |
| Talc | | 1.84 |
| Tablet weight | | 431.1 |

A double layer tablet (300 mg outer Immediate release layer with inner 300 mg matrix layer) is made. Alternately separate tablets are made and encapsulated in a hard gelatine tablet.

Bilayer tablet variants may be manufactured on a rotary multi-layer tablet press by filling the die step-wise with the contents of the two layers with subsequent compression into tablets. After the die is filled with the content of one layer, the tablelling punches compress the powder bed slightly before the die is additionally filled with the content of the succeeding layer and final compression leading to a bi-layer tablet.

Examples of typical release rates are

4 hours from 37 to 57%, e.g 49%

15 8 hours from 66 to 80%,e.g 73%

under vitro oxcarbazepine test dissolution conditions of the invention

### EXAMPLE E

### Variant 1—Disintegrating Tablet with Immediate Granulate and Modified Release Granulate

| Formulation | (mg) |
|---|---|
| Tablet Core: | |
| Compound of the invention | 600.0 |
| Eudragit RL30D | 45.0 |
| Avicel PH 102 | 188.6 |
| Cellulose HPM 603 | 8.4 |
| Croscarmellose sodium (Na | 75.0 |
| CMC XL) | |
| Aerosil 200 | 2.8 |
| Magnesium stearate | 4.8 |
| Weight of the core | 924.6 |
| Coating: | |
| Iron Oxide Yellow | 0.87 |
| TiO2 | 1.30 |
| PEG4000 | 1.74 |
| Cellulose HPM 603 | 17.36 |
| Talc | 3.04 |
| Tablet weight | 948.92 |

a) A pre-mix of 300 mg of the compound of the invention, dry substance of Eudragit RL30D, Avicel PH102 and cellulose HPM 603 was prepared.
b) A pre-mix of 300 mg of the compound of the invention, Avicel and cellulose HPM 603 was prepared.
c) Avicel PH 102, croscarmellose sodium and Aerosil 200 were screened and mixed using a bin blender (e.g. Turbula) and mixed with pre-mix a) and pre-mix b)
d) Magnesium stearate was screened through a hand screen (0.8 mm mesh) and added.
e) The final blend is mixed using a bin blender (e.g. Turbula).

The final blend may be compressed using e.g. a Killian LX18 tabletting press. The tablets may be filmed using a pan coater e.g. Glatt GC750.

Examples of typical release rates are

2 hours from 93 to 98%, e.g 95%

under vitro oxcarbazepine test dissolution conditions of the invention

### EXAMPLE F

### Variant 2; Slow Variant with Surfactant; 80% Release After 7-8 Hours

| Formulation | (mg) |
|---|---|
| Tablet Core: | |
| Compound of the invention | 600.0 |
| Duponol C | 6.0 |
| Avicel PH 102 | 125.2 |
| Cellulose HPM 603 | 16.8 |
| Cellulose HPM 100T | 73.8 |
| Aerosil 200 | 3.0 |
| Magnesium stearate | 8.0 |
| Weight of the core | 832.8 |
| Coating: | |
| Iron Oxide Yellow | 0.82 |
| TiO2 | 1.23 |
| PEG4000 | 1.64 |
| Cellulose HPM 603 | 16.41 |
| Talc | 2.88 |
| Tablet weight | 855.785 |

a) A pre-mix of the compound of the invention, Avicel PH, Cellulose HPM 603 and 6 mg of Duponol C (Sulfuric acid monododecyl ester sodium salt) was prepared.
b) Purified water is added to the pre-mix and granulated using a high-shear mixer (e.g. Aeromatic GP65).
c) The resulting granulation was screened using a Quadracomill and
d) dried using a fluid bed dryer (e.g. Aeromatic MP3/4).
e) Avicel PH102, HPM-Cellulose 100T and Aerosil 200 were screened with the dried granulation using a mill (e.g Frewitt) equipped with 1 mm mesh and mixed using a bin blender (e.g. Turbula).
f) Magnesium stearate is screened through a hand screen (0.8 mm mesh) and added.
g) The final blend is mixed using a bin blender (e.g. Turbula).

The final blend may be compressed using e.g. a Killian LX18 tabletting press. The tablets may be filmed using a pan coater e.g. Glaft GC750.

Examples of typical release rates are

4 hours from 30 to 52%, e.g 40%

8 hours from 47 to 75%, e.g 61%

under in vitro oxcarbazepine test dissolution conditions of the invention

Realease rate 80% in 12 hours

TABLE A
MHD levels

| | | | Ratio | | | | | | | | | |
|---|---|---|---|---|---|---|---|---|---|---|---|---|
| | AUC 0-48 | | Fed/ | Tmax | | Ratio | Cmax | | Ratio | C_{24 h} | | Ratio |
| Treat- | Fasted | Fed | Fast- | Fast- | | Fed/ | Fasted | Fed | Fed/ | Fasted | Fed | Fed/ |
| ment | (h * micromol/ | (h * micromol/ | ed | ed | Fed | Fasted | (micromol/ | (micromol/ | Fasted | (micromol/ | (micromol/ | Fasted |
| No | L) | L) | (%) | (h) | (h) | (%) | L) | L) | (%) | L) | L) | (%) |
| A | 740 | 698 | 0.94 | 20.4 | 6.6 | 0.32 | 23.1 | 34.3 | 1.49 | 22.4 | 15.9 | 0.71 |
| B | 539 | 459 | 0.85 | 8 | 5.4 | 0.68 | 19.9 | 22 | 1.11 | 13.9 | 9.44 | 0.68 |
| C | 437 | | | 6.8 | | | 15.9 | | | 10.9 | | |
| D | 681 | 771 | 1.13 | 6.2 | 5 | 0.81 | 24.3 | 35.7 | 1.47 | 17.2 | 18.0 | 1.04 |
| E | 548 | 771 | 1.41 | 4.4 | 6.8 | 1.55 | 26.9 | 37.6 | 1.40 | 11.9 | 16.1 | 1.35 |
| F | 410 | | | 25.6 | | | 13.9 | | | 13.5 | | |
| G | 749 | | | | | | | | | 22.9 | | |
| H | 675 | | | 5.2 | | | 31.8 | | | 13.7 | | |

G = film-coated tablet immediate release (Trileptal) 300 mg bid
H = film-coated tablet immediate release (Trileptal) 600 mg as a single dose

TABLE B
(Based on results of Table A)

| | Cmax | C_{24 h} | |
|---|---|---|---|
| Treatment | Fasted | Fasted | Ratio |
| No | (micromol/L) | (micromol/L) | Cmax/C_{24 h} |
| A | 23.1 | 22.4 | 1.03 |
| B | 19.9 | 13.9 | 1.43 |
| C | 15.9 | 10.9 | 1.46 |
| D | 24.3 | 17.2 | 1.41 |
| E | 26.9 | 11.9 | 2.26 |
| F | 13.9 | 13.5 | 1.03 |
| G | | 22.9 | |
| H | 31.8 | 13.7 | 2.32 |

1. An oral dosage form comprising oxcarbazepine adapted to be administered once a day.

2. The oral dosage form comprising oxcarbazepine according to claim 1 which, when administered once a day, is released to produce constant MHD plasma levels over 24 hours.

3. The oral dosage form according to claim 1 consisting of a tablet core and a coating wherein the core comprises oxcarbazepine, optionally, a filler, and at least one further excipient selected from the group comprising cellulose ethers, carboxyvinyl polymer of acrylic acid cross linked with alkyl ethers of sucrose, carboxyvinyl polymer of acrylic acid cross linked with alkyl ethers of pentaerythritol and polymethacrylates.

4. The oral dosage form according to claim 3 wherein said cellulose ether is hydroxypropyl methyl cellulose.

5. The oral dosage form according to claim 4 wherein the weight ratio of total hydroxypropyl-methyl cellulose to oxcarbazepine is from about 1:10 to about 1:20.

6. The oral dosage form according to claim 3 wherein said cellulose ether ethyl cellulose.

7. The oral dosage form according to claim 6 wherein the weight ratio of total ethyl cellulose to oxcarbazepine is from about 1:10 to about 1:20.

8. The oral dosage form according to claim 3 comprising a polymethacrylate which is trimethylammonium methacrylate.

9. The oral dosage form according to claim 3 wherein said filler is microcrystalline cellulose.

10. The oral dosage form according to claim 1 wherein said dosage form has an a 80% or greater release of the oxcarbazepine dose within 1 hour indicated in standard in vitro dissolution tests at 37 degrees Celsius in water using sodium dodecyl sulphate as a solubilizing agent at a concentration of 1% for a 600 mg dosage form.

11. The oral dosage form according to claim 1 wherein said dosage form releases oxcarbazepine at a constant release rate for 4 hours or more as indicated in standard in vitro dissolution tests at 37 degrees Celsius in water using sodium dodecyl sulphate as a solubilizing agent at a concentration of 1% for a 600 mg dosage form.

12. The oral dosage form according to claim 11 wherein said dosage form releases about 80% of oxcarbazepine within 8 hours.

13. An oral dosage form comprising oxcarbazepine which, when administered once a day, is released to produce constant MHD plasma levels over 24 hours.

14. (canceled)

15. (canceled)

16. A method for the treatment of epilepsy comprising orally administering to a patient in need of oxcarbazepine an oral dosage form of claim 1.

17. A method of reducing the variability of bioavailability of cyclosporin A for patients during oxcarbazepine therapy, said method comprising orally administering to a patient in need of oxcarbazepine therapy an oral dosage form of claim 1.
